(19) United States
(12) Patent Application Publication
(10) Pub. No.: US 2021/0245209 A1
(43) Pub. Date: Aug. 12, 2021

(54) TEXTILE THREAD ELEMENT FOR TREATING THE INSIDE OF A TUBE COMPRISING RIGID AND FLEXIBLE PORTIONS

(71) Applicants: COMMISSARIAT A L'ENERGIE ATOMIQUE ET AUX ENERGIES ALTERNATIVES, Paris (FR); STN, Comines (FR)

(72) Inventors: Frédérik GUYON, Leognan (FR); Guillaume NAVARRO, Cestas (FR); Mathias FAUCHILLE, Comines (FR)

(73) Assignees: COMMISSARIAT A L'ENERGIE ATOMIQUE ET AUX ENERGIES ALTERNATIVES, Paris (FR); STN, Comines (FR)

(21) Appl. No.: 17/273,378

(22) PCT Filed: Sep. 2, 2019
(86) PCT No.: PCT/FR2019/052015

§ 371 Date: Mar. 4, 2021

(30) Foreign Application Priority Data
Sep. 6, 2018 (FR) 1858019

(51) Int. Cl. B08B 9/043; D04C 1/02

(52) U.S. Cl. CPC B08B 9/0436; B08B 2209/04; D04C 1/02

(57) ABSTRACT

A textile thread element for treating a tube includes a first portion, called the “rigid portion”, forming a thread puller for introducing and front traction of the thread element inside the tube and a second portion, called the “flexible portion”, forming a flexible thread for treating the inside of the tube. The rigidity of the first portion is greater than that of the second portion. The first portion and the second portion are made from the same material or materials and are continuous with each other.

### TECHNICAL FIELD

The present invention relates to the general field of treating the inside of tubes of millimetric diameter, this treating comprising in particular the running-in, the polishing and/or the cleaning of the inside of tubes.

More precisely, the invention relates to a textile thread element comprising a first rigid portion and a second flexible portion, a unit comprising such a thread element and a tube into which the thread element is introduced, as well as a method of manufacturing such a thread element.

### PRIOR ART

Throughout the description, the expression “treating the inter-tube” designates one or more operations of running-in, polishing and/or cleaning the inside of a tube of millimetric diameter. In addition, the term “millimetric diameter” means a diameter of about a few tens of millimetres to a few millimetres.

The treating of the inter-tube of millimetric diameter over great lengths, typically of about several tens of centimetres, even more than a metre, is a source of difficulties with the solutions known from the prior art. The latter conventionally comprise: swabs; rigid shafts coated or not with suitable material; a string or rubbing fabric of close diameter, even slightly greater, than the inter-tube, introduced beforehand using a thread puller.

Regarding the swabs, their head is either made of a metallic material, which makes it possible to effectively detach dirty deposits but results in a risk of scratching the tube, or made of a polymer material, which does not scratch the tube but which has only very limited effectiveness on adherent dirt. In addition, the polymer material is fragile and wears quickly to the point of no longer being able to provide the friction necessary for the various treatments inside the tube. Moreover, the shaft of the swabs is made of a metallic material, which is required for the stiffness and the transmission of the torsion torque in the rotation movement of the swab. This shaft, which can be in contact with the tube of great length, can therefore also scratch the inside of the tube.

Regarding rigid shafts coated or not with a suitable material, these are shafts that are sufficiently rigid to withstand the torsion and compression/traction forces linked to the back-and-forth movements and rotation required for the operations of treating the inter-tube, in other words to the operations of running-in, polishing and/or cleaning. By way of examples, the materials used for such rigid shafts can comprise wood or metal. The shafts can for example be single-strand or multi-strand metallic shafts coated with suitable material, such as an abrasive material for running-in or polishing, or a soft material for cleaning. Also note that, for diameters of the inter-tube of about a millimetre, there are metal shafts or cables that can transmit these forces, but this solution is suitable for a rough running-in or polishing without therefore allowing for a fine running-in or polishing, or where applicable a cleaning without deterioration of the internal surface state of the tube.

Concerning the string or rubbing fabric, it is a textile, synthetic or natural material, which is pulled by force inside the tube. One of the difficulties of use is based on the introducing of this string of a nominal diameter that is necessarily close to, even slightly greater, than the inner diameter of the tube. To do this, a small-diameter thread puller is used, made of polymer or metal material that is lined so as to not scratch the tube. The thread puller is then passed first, then a mechanical connection is made with the textile material. Finally, via traction on the thread puller, the textile material is drawn progressively through the tube. This solution works for tubes with an inner diameter of a few millimetres, for example for an inner diameter of 2.5 mm or more. However, it is very delicate to use for long tubes with smaller inner diameters. Moreover, note that there are thread pullers and textile materials that sufficiently resistant to withstand the forces, but all the difficulty resides in the fragility of the connection between thread puller and textile material: in particular, either this connection imposes an excessive diameter; or it imposes radiuses of curvature that are too low which do not withstand the forces; or it imposes a thread puller and/or a textile material with a diameter that is too small to be functional or to withstand the traction forces.

DISCLOSURE OF THE INVENTION

The invention this has for purpose to overcome at least partially the needs mentioned hereinabove and the disadvantages concerning the embodiments of the prior art.

The invention also has for object, according to one of its aspects, a textile thread element for treating a tube, in particular a tube of millimetric diameter, characterised in that it comprises:

a first portion, called the “rigid portion”, forming a thread puller for introducing and front traction of the thread element inside the tube,

a second portion, called “flexible” portion, forming a flexible thread for treating the inside of the tube,

the rigidity of the first portion being greater than that of the second portion, and the first portion and the second portion being made from the same material or materials and continuous with each other.

The term “rigid” means that the first portion can be rigid or semi-rigid.

In addition, note that the second portion may also be used for the front traction of the thread element inside the tube, in particular in case of a complete introduction inside the tube and exceeding on either side of the tube.

Advantageously, the first and second portions are continuous with one another. In other words, there is no discontinuity between the two portions and no mechanical connection is required to connect them together. The first and second portions are advantageously formed from the same piece. They are advantageously connected together continuously, with the absence of any junction.

Thanks to the invention, it can be possible to carry out the functionality of thread puller and the functionality of treating the inter-tube via an identical textile material or materials thus suppressing the need for a mechanical connection inherent in heterogenous solutions of the prior art which call for an initial separation between thread puller and textile material.

The thread element according to the invention can further comprise one or more of the following characteristics taken individually or in any technically permissible combinations.

The second portion can include, over its length, one or more geometrical variations at the surface, in particular one or more asperities on the surface thereof, for example reliefs and/or hollows, used for treating the inside of the tube.

The first portion can advantageously be, over its length, substantially uniform.

Moreover, the thread element according to the invention can be made at least partially, in particular entirely, from thermosetting fibres.

The thread element can include threads dedicated to specific functions. In particular, the second portion can include threads dedicated to specific functions chosen from fluid absorption fibres and/or abrasion fibres of the inside of the tube and/or metallic threads, among others, which are advantageously non-thermosettable.

Moreover, the thread element can furthermore include a third portion, called “flexible or rigid portion”, forming a gripper thread for the rear traction of the thread element inside the tube. The third portion and the second portion can be made of the same material or materials and continuous with each other.

Advantageously, the third, second and first portions are continuous with each other. In other words, there is no discontinuity between the three portions and no mechanical connection is required to connect them together. The first, second and third portions are advantageously formed from a single piece.

In addition, only the second portion can include an abrasive coating.

Advantageously, when the thread element includes only a first portion and a second portion, in other words no third portion, the second portion can former a gripper thread for the rear traction of the thread element inside the tube. When the thread element comprises first, second and third portions, the third portion is the preferred portion for the rear traction of the thread element. Where applicable, in this second configuration, the second portion can also be used for the rear traction.

Furthermore, the invention further has for object, according to another of its aspects, a unit, characterised in that it comprises:

a textile thread element such as defined hereinabove,

a tube, in particular a tube of millimetric diameter, wherein the thread element is introduced for treating the inside of the tube.

The length of the thread element can preferably be greater than the length of the tube.

Furthermore, the length of the first rigid portion can advantageously be greater than the length of the tube.

Moreover, the invention also has for object, according to another of its aspects, a method for manufacturing a textile thread element such as defined hereinabove, characterised in that it comprises the following steps:

braiding, in particular continuously, of textile filaments to form the thread element,

heating, in particular intermittently, of the first portion to render it rigid.

Advantageously, when the thread element comprises at least partially, in particular entirely, thermosetting fibres, the method according to the invention can comprise the step consisting of thermosetting, in particular by intermittent heating, the first portion to render it rigid.

The thread element, the unit and the method of manufacturing according to the invention can comprise any of the characteristics mentioned in the description, taken individually or according to any technically permissible combinations with other characteristics.

### BRIEF DESCRIPTION OF THE DRAWINGS

The invention can be understood better when reading the following detailed description, of examples of implementations that do not limit the latter, as well as when examining figures, diagrammatical and partial, of the accompanying drawing, wherein:

FIG. 1 shows an example of a textile thread element in accordance with the invention,

FIG. 2 shows an example of a unit comprising a tube of millimetric diameter and a textile thread element in accordance with the invention, such as that of FIG. 1,

FIG. 3 shows a method for manufacturing a textile thread element in accordance with the invention, such as that of FIG. 1, and

FIG. 4 shows another example of a textile thread element in accordance with the invention.

The different portions shown in these figures are not necessarily shown according to a uniform scale, in order to make the latter more legible.

### DETAILED DISCLOSURE OF PARTICULAR EMBODIMENTS

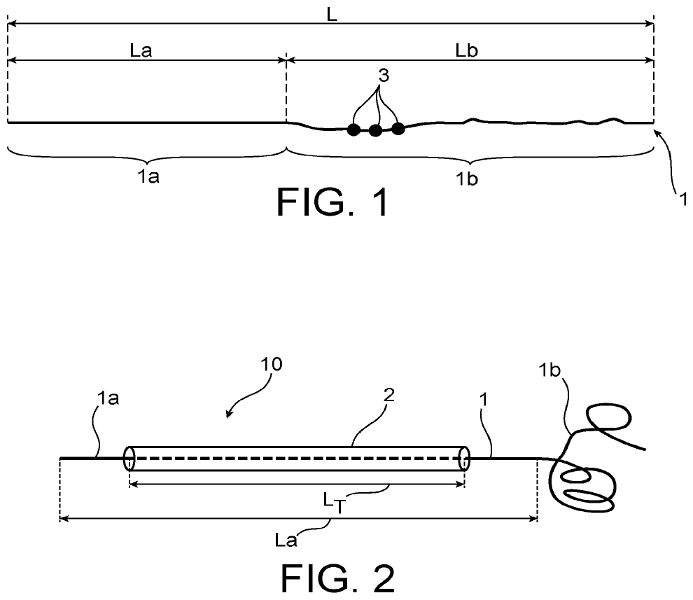

In reference to FIGS. 1 to 3, an example of a textile thread element 1 in accordance with the invention shall be described hereinafter for the treating the inside of a tube 2 of millimetric diameter.

This textile thread element 1 generally has the form of a string 1 such as shown in FIG. 1.

In accordance with the invention, the string 1 comprises a first portion 1a called the “rigid portion”, and a second portion 1b called “flexible” portion.

The string 1 is homogeneous because the rigid 1a and flexible 1b portions are made from the same material or materials, and are continuous with each other. In other words, except concerning the rigidity and possibly the geometrical variations, there is no difference between the rigid 1a and flexible 1b portions. They are made from a single piece and together define the string 1.

The first rigid portion 1a is more particularly rigid or semi-rigid. It is qualified as “rigid” because it has a rigidity that is more substantial than the second portion 1b called “flexible”.

More precisely, the string 1 thus has a dual functionality, namely that of a thread puller and that of treating the inter-tube, as explained hereinabove in the part relative the prior art.

Thus, the rigid portion 1a has a rigidity such as that usually used for a thread puller used for the principle if strings or rubbing fabrics. This rigid portion 1a allows for the introduction of the string 1, and in particular of the flexible portion 1b, into the tube 2.

The flexible portion 1b has a flexibility such as that usually used for a string or conventional rubbing fabric. This flexible part 1b makes it possible to treat the inside of the tube 2.

FIG. 2 shows the insertion of the string 1 of FIG. 1 into the tube 2 of millimetric diameter. More particularly, this can be a tube 2 with an inner diameter of about 1 mm and a length L_{T} of about 1,500 mm.

Advantageously, the length L of the string 1 is greater than the length L_{T} of the tube 2. Even more precisely, the length La of the first rigid portion 1a is greater than the length L_{T} of the tube 2. In this way, the rigid portion 1a is entirely introduced into the tube 2, a portion of the latter exceeding the tube 2 at each of the ends thereof as can be seen in FIG. 2. Then, the flexible portion 1b of the string 2, visible on the right in FIG. 2, outside the tube 2, is ready to be pulled in the tube 2 in order to allow for the treating thereof.

Moreover, as can be seen in FIG. 1, the rigid portion 1a has an aspect, along its length La, substantially uniform in such a way as to facilitate the introduction thereof into the tube 2 and no damage the inside of the tube 2.

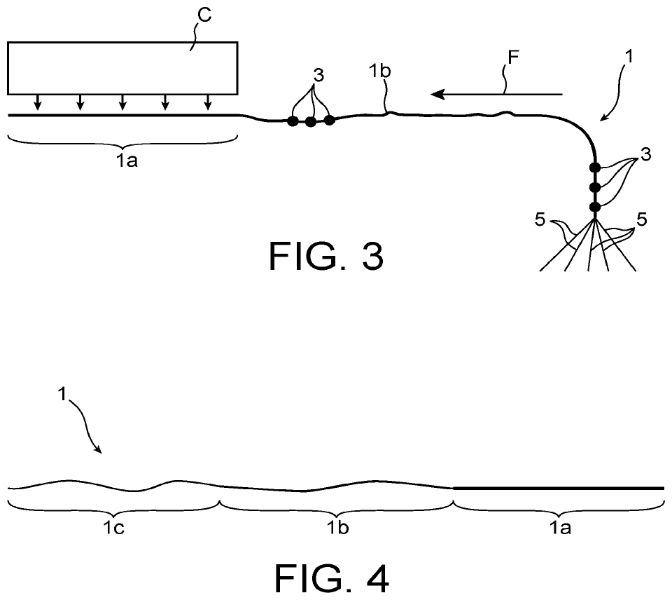

On the contrary, as can be seen in FIGS. 1 and 3, the flexible portion 1b of the string 1 can include, along the length Lb thereof, geometrical variations 3 on the surface, here for example reliefs 3 with a spherical shape, used for treating the inside of the tube 2. More precisely, the string 1 includes one-off modifications in geometry to functionalise the flexible portion 1b dedicated for treating the inter-tube, for example surface asperities, in particular extra thicknesses, abrasives and/or cavities to for example rub the inner wall of the tube 2 or maintain cleaning fluids.

Furthermore, the string 1 is advantageously made with thermosetting fibres so as to allow for the formation of the rigid portion 1a.

More precisely, the string 1 is made, providing the two functions of thread puller and of treating the inter-tube, by thermosetting of a portion of the latter to form the rigid portion 1a, without thus needing a connection with the other portion forming the flexible portion 1b.

FIG. 3 diagrammatically shows a principle for manufacturing the string 1 in accordance with the invention.

The string 1 is thus formed, entirely or partially, by a conventional method of braiding continuously from thermosetting fibres, the arrow F representing the direction of travel in FIG. 3.

Thus, textile filaments 5 are braided to form the string 1 in the direction of the arrow F in FIG. 3.

The rigid portion 1a, dedicated to the function of thread puller, is formed by thermosetting over a length La equal at least to the length L_{T} of the tube 2 to be treated supplemented by an extra length for the gripping and the inserting, by intermittent heating C of the portion of the string 1 formed first in the method of braiding.

The other portion, intended to form the flexible portion 1b, is not heated to maintain the flexibility but is provided with geometrical variations 3 intended to improve the treating of the inter-tube.

The thermosetting operation can be carried out during the travel of the string 1 at the output of the operation of braiding, as shown in FIG. 3, or in a second step, after a possible cutting into multiple lengths of the length of the final use of the string 1.

Moreover, the string 1 can also comprise, in addition to thermosettable fibres, other types of fibres to provide the string 1 with other properties, for example fibres for absorbing or for retaining cleaning fluids and/or abrasion fibres of the inside of the tube 2 and/or resistant fibres for the treating of the inter-tube.

FIG. 4 shows moreover another example of a string 1 in accordance with the invention.

In this example, as hereinabove, the string 1 comprises a first rigid portion 1a, and a second flexible portion 1b. The rigid portion 1a allows for the insertion and the front traction. The flexible portion 1b allows for the treating of the inside of the tube 2.

This flexible part 1b can have a first configuration similar to that described hereinabove, that may or may not comprise variations in geometry along the latter and can comprise threads dedicated to specific functions chosen from fluid absorption fibres and/or abrasion fibres of the inside of the tube 2 and/or metallic threads, the latter thus advantageously being non-thermosetting. It can also have a second configuration, non-exclusive from the first, wherein it alone is coated with an abrasive coating.

Moreover, in this example, the string 1 also comprises a third portion 1c, called “flexible or rigid portion”, forming a gripper thread for the rear traction of the thread element 1 inside the tube 2.

This third portion 1c can be flexible and thus form a specific portion, or be semi-rigid forming the beginning of the following section.

Of course, the invention is not limited to the embodiments that have just been described. Various modifications can be made thereto by those skilled in the art.

1-13. (canceled)

14. A textile thread element for treating a tube, comprising:
a first portion, called a “rigid portion”, forming a thread puller for introducing and front traction of the thread element inside the tube; and
a second portion, called a “flexible portion”, forming a flexible thread for treating the inside of the tube,
the rigidity of the first portion being greater than that of the second portion, and the first portion and the second portion being made from the same material or materials and continuous with each other.

15. The thread element according to claim 14, wherein the second portion comprises, over its length, one or more geometrical variations on the surface used for treating the inside of a tube.

16. The thread element according to claim 14, wherein the first portion is, over its length, uniform.

17. The thread element according to claim 14, wherein the thread element is made at least partially from thermosetting fibres.

18. The thread element according to claim 14, wherein the second portion comprises threads dedicated to specific functions chosen from at least one of fluid absorption fibres, abrasion fibres of the inside of the tube, and metallic threads.

19. The thread element according to claim 14, further comprising:
a third portion, called “flexible or rigid portion”, forming a gripper thread for the rear traction of the thread element inside the tube, the third portion and the second portion being made from the same material or materials and continuous with each other.

20. The thread element according to claim 14, wherein only the second portion comprises an abrasive coating.

21. The thread element according to claim 14, wherein the first and second portions are made from a single piece, being continuously connected together, with the absence of any junction.

22. A unit, comprising:
the textile thread element according to claim 14; and
a tube, wherein the thread element is introduced for treating the inside of the tube.

23. The unit according to claim 22, wherein the length of the thread element is greater than the length of the tube.

24. The unit according to claim 22, wherein the length of the first rigid portion is greater than the length of the tube.

25. A method for manufacturing the textile thread element according to claim 14, comprising:
braiding textile filaments to form the thread element; and
heating of the first portion to render the first portion rigid.

26. The method according to claim 25, wherein, the thread element comprises at least partially thermosetting fibres, and the method further comprises thermosetting the first portion to render the first portion rigid.
